(19) United States
(12) Patent Application Publication
(10) Pub. No.: US 2022/0301701 A1
(43) Pub. Date: Sep. 22, 2022

(54) SYSTEMS AND METHODS TO DYNAMICALLY DISTRIBUTE THERAPEUTIC SUPPORT SURFACES

(71) Applicant: JOERNS HEALTHCARE, LLC, CHARLOTTE, NC (US)

(72) Inventors: William H. NELSON, Venetia, PA (US); John Douglas FERGUSON, Monroe (CA)

(21) Appl. No.: 17/207,414

(22) Filed: Mar. 19, 2021

(51) Int. Cl. G16H 40/40; G16H 10/60

(52) U.S. Cl. CPC G16H 40/40; G16H 10/60

(57) ABSTRACT

Systems and methods to dynamically distribute therapeutic support surfaces are provided. The method includes receiving a request to provide a patient with a therapeutic support surface. The method also includes obtaining a plurality of medical records of the patient. The method further includes determining a designated location, at which, the patient is expected to occupy the therapeutic support surface, and a period of time, during which, the patient is expected to occupy the therapeutic support surface. The method further includes determining, based on the plurality of medical records, the designated location, and the period of time, an available therapeutic support surface for the patient. The method further includes requesting the available therapeutic support surface to be dispatched to the designated location within a second period of time prior to an estimated arrival of the patient to the designated location.

### BACKGROUND

The present disclosure relates generally to systems and methods to dynamically distribute therapeutic support surfaces.

Therapeutic support surfaces are sometimes provided to acute care, post-acute care, and other types of healthcare facilities to provide comfort to patients during their stay at the facilities. However, due to fluctuations in the intake of patients having different medical conditions, healthcare facilities sometimes lack the number of therapeutic support surfaces to accommodate patients, or lack suitable therapeutic support surfaces to accommodate patients having particular medical conditions. Alternatively, healthcare facilities sometimes have an excess of therapeutic support surfaces that are not being occupied while other healthcare facilities are in need of the excess therapeutic support surfaces.

### BRIEF DESCRIPTION OF THE DRAWINGS

Illustrative embodiments of the present invention are described in detail below with reference to the attached drawing Figures, which are incorporated by reference herein, and wherein:

FIG. 1A is a network environment for dynamically distributing therapeutic support surfaces in accordance with one embodiment.

FIG. 1B is another network environment for dynamically distributing therapeutic support surfaces in accordance with one embodiment.

FIG. 2 is a tree diagram illustrating a process to select a therapeutic support surface in accordance with one embodiment.

FIG. 3 is a system diagram of the therapeutic support surface distribution system of FIGS. 1A and 1B in accordance with one embodiment.

FIG. 4 is a flowchart of a process to dynamically distribute therapeutic support surfaces in accordance with one embodiment.

The illustrated figures are only exemplary and are not intended to assert or imply any limitation with regard to the environment, architecture, design, or process in which different embodiments may be implemented.

### DETAILED DESCRIPTION

In the following detailed description of the illustrative embodiments, reference is made to the accompanying drawings that form a part hereof. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments is defined only by the appended claims.

The present disclosure relates to systems and methods to dynamically distribute therapeutic support surfaces. As referred to herein, a therapeutic support surface refers to any bed, chair, recliner, standing chair, lift, furniture, or equipment having a surface that a patient occupies during acute care, post-acute care, and/or home care. A request to provide a patient with a therapeutic support surface is received by a therapeutic support surface distribution system. In some embodiments, the request is transmitted from a healthcare facility, such as an acute care facility, a post-acute care facility, or another type of healthcare facility prior to or during patient intake. In some embodiments, the request is transmitted by an individual, such as a caretaker, or by the patient. The therapeutic support surface distribution system obtains medical records (e.g., electronic health records) of the patient, such as, but not limited to, prior and existing medical conditions of the patient and one or more medical preferences of the patient. The therapeutic support surface distribution system also determines a designated location at which the patient is expected to occupy the therapeutic support surface, and a period of time the patient is expected to occupy the therapeutic support surface. Examples of designated locations include, but are not limited to, acute care facilities, post-acute care facilities, nursing homes, patient homes, and other locations at which the patient is expected to occupy the therapeutic support surface. In some embodiments, the therapeutic support surface distribution system obtains a detailed description of the designated location, such as the room number of a post-acute care facility the patient is expected to reside while occupying the therapeutic support surface.

The therapeutic support surface distribution system selects an available therapeutic support surface based on the patient's records, the designated location, and the period of time the patient is expected to occupy the therapeutic surface. As referred to herein, an available therapeutic support surface is a therapeutic support surface that is available to the patient during the period of time the patient is expected to occupy the therapeutic support surface. In some embodiments, a therapeutic support surface is categorized as an available therapeutic support surface even if it is being occupied by another patient at the time the therapeutic support surface distribution system processes a request so long as there is sufficient time to transport and set up the therapeutic support surface at the designated location by the time the patient is expected to occupy the therapeutic support surface.

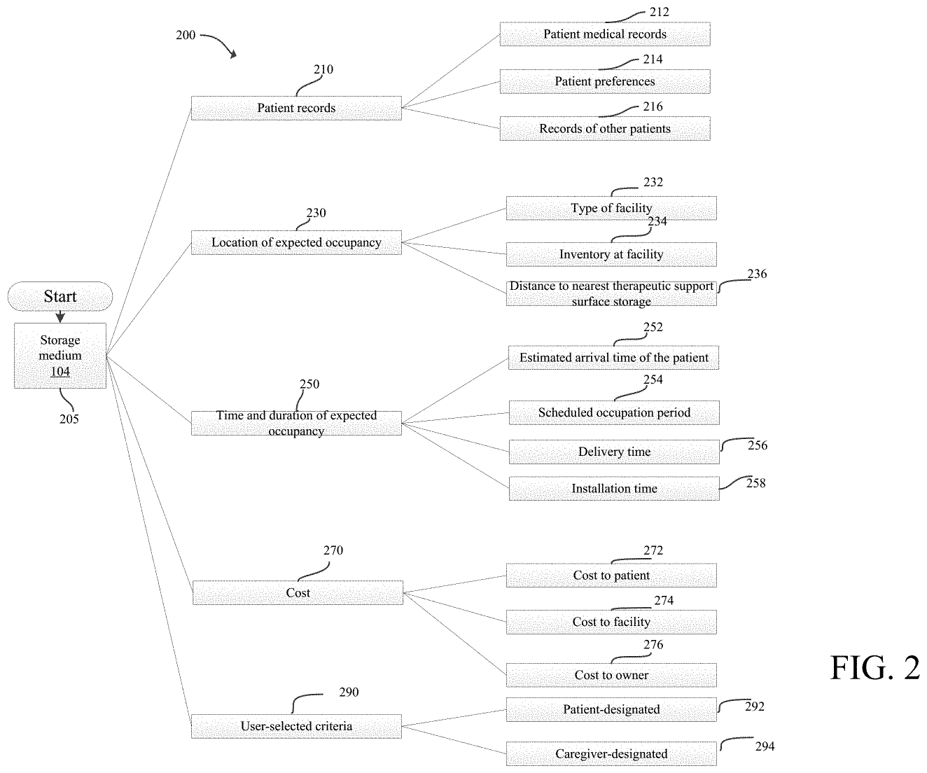

In some embodiments, the therapeutic support surface distribution system selects an available therapeutic support surface based on additional criteria, such as, but not limited to, preferences of the patient, other patients with similar medical conditions, type of the facility, the current inventory of therapeutic support surfaces, distance from the facility to the nearest therapeutic support surface storage location, the delivery time to the patient, the delivery time to the next patient, the installation time, the cost to the patient, the cost to the facility, and the owner of the therapeutic support surface. Additional descriptions of operations performed by the therapeutic support surface distribution system to select a therapeutic support surface are provided herein and are illustrated by at least FIG. 2, which illustrates a tree diagram of a process to select a therapeutic support surface. As referred to herein, a therapeutic support surface storage location refers to any location where one or more therapeutic support surfaces are stored. Examples of therapeutic support surface storage locations include, but are not limited to, warehouses, medical facilities, patient homes, and other locations where therapeutic support surfaces are stored.

In some embodiments, the therapeutic support surface distribution system provides an user, such as a caregiver or the patient, with one or more user-selected choices to select a therapeutic support surface from an inventory of therapeutic support surfaces. In one or more of such embodiments, the number of types of modifications and choices available to the user are based on the inventory, the medical condition of the patient, and/or therapeutic support surfaces available to the patient.

The therapeutic support surface requests the available therapeutic support surface to be dispatched from the storage location to the designated location within a period of time prior to an estimated time of arrival of the patient to the designated location (e.g., one hour before the patient is expected to arrive at a post-acute care facility). In some embodiments, the therapeutic support surface distribution system requests the available therapeutic support surface to be immediately dispatched to the designated location. In some embodiments, the therapeutic support surface distribution system requests the therapeutic support surface to be dispatched from the storage location at a designated time (e.g., on Jan. 5, 2021 at 12 pm Central Time) such that the therapeutic support surface will arrive at the designated location within a period of time before the time at which the patient is expected to arrive at the designated location.

In one or more of such embodiments, the therapeutic support surface distribution system dynamically monitors weather and traffic congestions within a geographic region (e.g., zip code, city, state, etc.), and periodically readjusts the time the therapeutic support surface is dispatched from the storage location, and the routes taken to transport the therapeutic support surface from the storage location to the designated location. In some embodiments, where the available therapeutic support surface is being occupied by another patient at the time it is selected by the therapeutic support surface distribution system, the therapeutic support surface distribution system dynamically requests and schedules retrieval of the available therapeutic support surface, cleaning and decontamination of the available therapeutic support surface, and delivery of the available therapeutic support surface to the designated location within a period of time before the patient is scheduled to arrive at the designated location. In some embodiments, the therapeutic support surface distribution system also dynamically schedules and requests modifications to the available therapeutic support surface based on one or more requests or preferences of the patient, the patient's caregiver, or the designated location. For example, the therapeutic support surface distribution system adjusts the pressure of a mattress of the therapeutic support surface to match the patient's preferred pressure, and adjusts the wheels of the therapeutic support surface to comply with a request from the facility at which the designated location is located for easier movement around the facility.

In some embodiments, the therapeutic support surface periodically determines the availability of the therapeutic support surface due to one or more patient related or non-patient related (e.g. weather) changes. In one or more of such embodiments, where the therapeutic support surface determines that the available therapeutic support surface becomes unavailable (e.g., cannot be delivered to the designated location in time), the therapeutic support surface distribution system dynamically searches an inventory of therapeutic support surfaces for similar or identical therapeutic support surfaces for delivery to the designated location. In one or more of such embodiments, where the therapeutic support surface distribution system determines that the inventory does not contain any identical therapeutic support surface or no identical therapeutic support surface is available for delivery to the designated location before the patient's arrival to the designated location, the therapeutic support surface dynamically requests and schedules one or more modifications to be made to similar therapeutic support surfaces to match some or all of the specifications of the unavailable therapeutic support surface.

In some embodiments, the therapeutic support surface distribution system, upon a determination of a change in the estimated arrival time of the patient to the designated location, or a new period of time the patient is scheduled to occupy the available therapeutic support surface, reschedules retrieval time of the available therapeutic support surface from the storage location to deliver the available therapeutic support surface within a period of time of the new estimated arrival time, and reschedules the retrieval time of the available therapeutic support surface from the designated location. Similarly, in some embodiments, where the therapeutic support surface distribution system determines a change to the period of time the patient is scheduled to occupy the distribution support surface, the therapeutic support surface distribution system dynamically determines a new period of time the patient is excepted to occupy the available therapeutic support surface, and reschedules the retrieval time of the available therapeutic support surface from the designated location.

In some embodiments, while the patient is occupying the available therapeutic support surface, the therapeutic support surface distribution system receives a request to provide a second patient with a therapeutic support surface together with records of the second patient. In one or more of such embodiments, the therapeutic support surface distribution system determines a second designated location at which the second patient is expected to occupy a therapeutic support surface, and a period of time during which the second patient is expected to occupy the second therapeutic support surface. In one or more of such embodiments, the therapeutic support surface distribution system determines that the available therapeutic support surface meets the criteria for the second patient and is deliverable to the second designated location within a period of time before the estimated arrival of the second patient to the second designated location, and determines to provide the available therapeutic support surface to the second patient. The therapeutic support distribution system then requests the available therapeutic support surface to be dispatched to the second designated location within a period of time before the estimated arrival of the second patient.

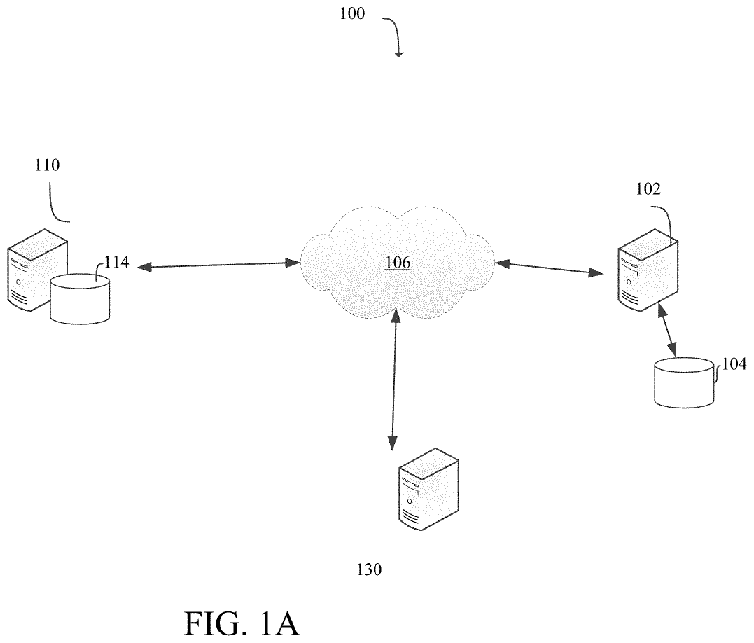

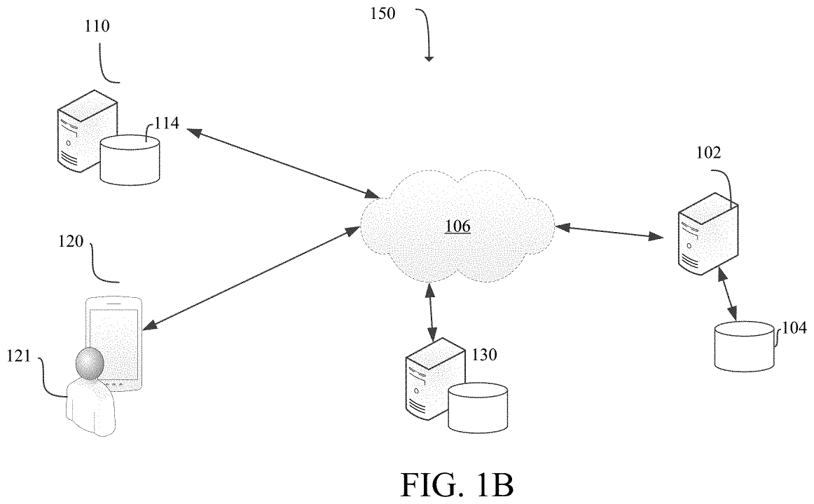

In some embodiments, the therapeutic support surface distribution system periodically determines a status of one or more conditions of the patient while the patient occupies the therapeutic support surface, and determines whether the patient's condition has improved or has improved at a faster rate than a threshold rate (e.g., an average recovery time period) while occupying the therapeutic support surface. For example, where the patient is recovering from back surgery, and the average recovery time period for similar or identical back surgery procedures is three months, the therapeutic support surface distribution system determines that the patient condition has improved if the recovery time is shorter than or approximately equal to the average recovery time period. In one or more of such embodiments, the therapeutic support surface distribution system in response to a determination of an improvement of the condition while occupying the therapeutic support surface, designates the therapeutic support surface or the model of the therapeutic support surface suitable for occupancy by future patients that experience a similar condition as the condition of the patient. In some embodiments, the therapeutic support surface distribution system analyzes the recovery period of each patient while occupying a therapeutic support surface as well as patient and/or caregiver feedback regarding the therapeutic support surface. In one or more of such embodiments, the therapeutic support surface distribution system generates one or more recommendations to improve one or more components (e.g., adjust the length of the headboard) of the therapeutic support surface to address feedbacks, and to improve the therapeutic support surface for future use and/or to tailor the therapeutic support surface for a particular future use, such as to provide to a patient suffering from a particular medical condition. Additional descriptions of methods to dynamically distribute therapeutic support surfaces and therapeutic support surface distribution systems are provided in the paragraphs below and are illustrated in at least FIGS. 1A-4.

FIG. 1A is a network environment 100 for dynamically distributing therapeutic support surfaces in accordance with one embodiment. Network environment 100 includes a therapeutic support surface distribution system 102 that is communicatively connected to a healthcare facility system 110, and a third-party system 130 via a network 106.

As referred to herein, a healthcare facility system 110 is an electronic system of a facility configured to request a therapeutic support surface from therapeutic support surface distribution system 102. Examples of healthcare facility systems include, but are not limited to, electronic systems of acute care facilities, electronic systems of post-acute care facilities, and patient home electronic systems. Healthcare facility system 110 is an electronic system that may be formed from one or more work management stations, server systems, desktop computers, laptop computers, tablet computers, smartphones, smart watches, PDAs, as well as similar electronic devices having a processor operable to request a therapeutic support surface from therapeutic support surface distribution system 102.

Healthcare facility system 110 includes or is communicatively connected to a storage medium, such as storage medium 114 that stores information regarding healthcare facility system 110, such as the location of the facility, the facility's hours of operation, the facility's contact information, a layout of the facility, including a layout of the designated location within the facility, location of the designated location within the facility (e.g., room number of the designated location, floor number of the designated location, etc.), as well as other information related to the facility and specifications of the facility. Certain patient information, such as a patient's existing medical condition, the patient's estimated arrival time, the estimated duration of the patient's stay, and certain medical records of the patient are also stored in storage medium 114.

Healthcare facility system 110 transmits a request to provide the patient with a therapeutic support surface to therapeutic support surface distribution system 102 together with information regarding the patient, information regarding the designated location, at which the patient is expected to occupy the therapeutic support surface, information related to the estimated arrival time and length of stay at the facility including the length of time the patient is expected to occupy the therapeutic support surface, and other information related to delivery and installation of the therapeutic support surface. Therapeutic support surface distribution system 102 may be formed from one or more work management stations, server systems, desktop computers, laptop computers, tablet computers, smartphones, smart watches, PDAs, as well as similar electronic devices having one or more processor operable to receive a request for a therapeutic support surface together with patient information of the patient and delivery information, select an available therapeutic support surface, and request delivery of the therapeutic support surface. Additional descriptions of operations performed by therapeutic support surface distribution system 102 are provided herein and are illustrated in at least FIGS. 2-4. Therapeutic support surface distribution system 102 includes or is communicatively connected to a storage medium, such as storage medium 104. Storage medium 104 stores instructions, which when executed by one or more processors of therapeutic support surface distribution system 102, cause the processors to perform the foregoing operations as well as other operations described herein.

Storage medium 104 may be formed from data storage components such as, but not limited to, read-only memory (ROM), random access memory (RAM), flash memory, magnetic hard drives, solid state hard drives, CD-ROM drives, DVD drives, floppy disk drives, as well as other types of data storage components and devices. In some embodiments, storage medium 104 includes multiple data storage devices. In further embodiments, the multiple data storage devices may be physically stored at different locations. In one of such embodiments, the data storage devices are components of a server station, such as a cloud server. In another one of such embodiments, the data storage devices are components of therapeutic support surface distribution system 102. Storage medium 104, in addition to storing executable instructions, also stores information provided by healthcare facility system and by other third party systems, such as location and coordinates of the designated location, the patient's estimated time of arrival to the designated location, the patient's estimated time of stay at the healthcare facility, including the amount of time the patient is expected to occupy the therapeutic support surface, and certain patient records, to the extent storage of such patient records is permitted by law, and approved by the patient.

Therapeutic support surface distribution system 102 is also communicatively connected to one or more third-party systems or networks. Examples of third-party systems include weather station systems and networks, system and network of navigation systems, systems and networks of government agencies (such as, but not limited to local law enforcement agencies, federal law enforcement agencies, international government agencies, the federal emergency management agencies, and local emergency management agencies), social media networks, as well as other third-party systems and networks operable to provide up-to-date or forecasted data to therapeutic support surface distribution system 102. In the depicted embodiment, the third-party systems and networks are collectively illustrated as third-party system 130. In some embodiments, the third-party system 130 provides therapeutic support surface distribution system 102 with up-to-date information therapeutic support surface distribution system 102 utilizes to determine an estimated transmit time between a storage facility of an available therapeutic support surface and the designated location, as well as changes to the transit time due to weather, traffic, and/or other transit disruptions. In some embodiments, therapeutic support surface distribution system 102, in response to receiving a weather update from third-party system 130, determines an increase in the transit time from the storage location of the available therapeutic support surface to the designated location. In one or more of such embodiments, therapeutic support surface distribution system 102 dynamically requests the available therapeutic support surface to be transported from the storage location at an earlier time to account for the expected transit delay. In one or more of such embodiments, therapeutic support surface distribution system 102 dynamically searches an inventory of therapeutic support surfaces for a replacement therapeutic support surface (e.g., another available therapeutic support surface) that is stored at another storage location not affected or less affected by the transit disruption, and requests the replacement therapeutic support surface to be delivered from the other storage location to the designated location.

In some embodiments, therapeutic support surface distribution system 102 also dynamically or periodically communicates with healthcare facility system 110 regarding the status of the available therapeutic support surface, including the current location of the therapeutic support surface, expected delivery time, expected installation time, information regarding personnel and vehicle used to deliver the available therapeutic support surface, existence of potential delays, estimated length of such delays, and information regarding personnel and vehicle used to pick up the available therapeutic support surface. In some embodiments, therapeutic support surface distribution system 102 also dynamically communicates with personnel, including third-party personnel responsible for delivering the available therapeutic support surface, setting up the therapeutic support surface, and removing the therapeutic support surface.

In some embodiments, where healthcare facility system 110 also provides information regarding the patient's feedback regarding the therapeutic support surface or feedback from other personnel of the facility regarding the therapeutic support surface to therapeutic support surface distribution system 102, therapeutic support surface distribution system 102 utilizes such feedback to determine whether to provide similar or identical therapeutic support surfaces to future patients having similar or identical conditions. For example, where therapeutic support surface distribution system 102 receives a feedback from a patient recovering from a back surgery that the mattress was too soft, therapeutic support surface distribution system 102 will request therapeutic support surfaces having stiffer mattresses to be sent to future patients recovering from similar types of back surgeries. In one or more of such embodiments, where therapeutic support surface distribution system 102 receives a feedback while the patient is occupying the therapeutic support surface, therapeutic support surface distribution system 102 dynamically provides instructions to healthcare facility system 110 to request a personnel of the facility to make one or more adjustments to the configuration (e.g., increasing the pressure of the mattress to stiffen the mattress) of the therapeutic support surface to address the patient's feedback.

Network 106 can include, for example, any one or more of a cellular network, a satellite network, a personal area network (PAN), a local area network (LAN), a wide area network (WAN), a broadband network (BBN), a RFID network, a Bluetooth network, a device-to-device network, the Internet, and the like. Further, network 106 can include, but is not limited to, any one or more of the following network topologies, including a bus network, a star network, a ring network, a mesh network, a star-bus network, a tree or hierarchical network, or similar network architecture. Network 106 may be implemented using different protocols of the internet protocol suite such as TCP/IP. Network 106 includes one or more interfaces for data transfer. In some embodiments, network 106 includes a wired or wireless networking device (not shown) operable to facilitate one or more types of wired and wireless communication between therapeutic support surface distribution system 102, healthcare facility system 110, third-party system 130, as well as other electronic devices (not shown) and systems (not shown) communicatively connected to network 106. Examples of the networking device include, but are not limited to, wired and wireless routers, wired and wireless modems, access points, as well as other types of suitable networking devices described herein. Examples of wired and wireless communication include Ethernet, WiFi, Cellular, LTE, GPS, Bluetooth, and RFID, as well as other types of communication modes described herein.

FIG. 1B illustrates another network environment 150 for dynamically distributing therapeutic support surfaces in accordance with one embodiment. In environment 150, therapeutic support surface distribution system 102, storage medium 104, healthcare facility system 110, storage medium 114, and third-party system 130 are also communicated to each other via network 106. Further, an user 121 also communicates with therapeutic support surface distribution system 102 via an electronic device 120 to request therapeutic support surface distribution system 102 to provide a therapeutic support surface to a designated location. In some embodiments, user 121 is a personnel of the healthcare facility requesting the therapeutic support surface. In some embodiments, user 121 is the patient. Electronic device 120 includes any device configured to request a therapeutic support surface from therapeutic support surface distribution system 102. Examples of electronic device 120 include, but are not limited to, smartphones, tablet computers, desktop computers, laptop computers, smart watches, PDAs, as well as similar electronic devices having a processor operable to request a therapeutic support surface from therapeutic support surface distribution system 102, and is communicatively connected to a display.

In some embodiments, therapeutic support surface distribution system 102 determines, based on the patient's medical records, the designated location, and the patient's estimated arrival time and duration of stay, a set of user-selectable criteria for selecting the available therapeutic support surface, and provides the set of user-selectable criteria to electronic device 120. In some embodiments, user 121 operates electronic device 120 to provide additional instructions to therapeutic support surface distribution system 102 regarding one or more preferences or requirements of the patient, caregivers of the patient, and/or the healthcare facility. In some embodiments, certain requests made by user 121 via electronic device 120 supersedes incompatible requests made by healthcare facility system 110. For example, where user 121 requests a therapeutic support surface manufactured by a different manufacturer than the manufacturer selected by healthcare facility system 110, and provided all other considerations remain equal, then therapeutic support surface distribution system 102 will prioritize the user's selection over the selection made by healthcare facility system 110. In some embodiments, certain requests made by healthcare facility system 110 supersedes incompatible requests made by user 121. For example, where user 121 requests a therapeutic support surface that is not covered by the patient's insurance while healthcare facility system 110 selects another therapeutic support surface that is covered by the patient's insurance, then therapeutic support surface distribution system 102 will select the therapeutic support surface covered by the patient's insurance plan and notify user 121 via electronic device 120. In some embodiments, therapeutic support surface distribution system 102 does not provide certain incompatible options for display on electronic device 120. For example, models of certain therapeutic support surfaces that do not fit into the designated location are greyed out or are not provided as an available option to electronic device 120.

Although FIGS. 1A and 1B illustrate one healthcare facility system 110, in some embodiments, therapeutic support surface distribution system 102 receives requests from multiple healthcare facility systems (not shown) to provide therapeutic support surfaces to different healthcare facilities. In some embodiments, therapeutic support surface distribution system 102 dynamically performs operations described herein to concurrently communicate with multiple healthcare facility systems to fulfill requests to provide therapeutic support surfaces to the healthcare facilities, update and/or cancel existing requests, and to periodically provide delivery information to the healthcare facilities.

FIG. 2 is a tree diagram illustrating a process 200 to select a therapeutic support surface in accordance with one embodiment. Although operations in the process 200 are shown in a particular sequence, certain operations may be performed in different sequences or at the same time where feasible. Further, in some embodiments, different combinations of some or all of the steps of the process 200 are performed to select the therapeutic support surface.

At block 205, therapeutic support surface distribution system 102, upon receiving a request to provide a patient with a therapeutic support surface, accesses storage medium 104 to obtain instructions that define how to select a therapeutic support surface. In the depicted embodiment, instructions that define how to select a therapeutic support surface are categorized by several factors, including patient records, location of expected occupancy, time and duration of expected occupancy, cost, and user-selected criteria.

At block 210, therapeutic support surface distribution system 102 accesses a first category of instructions that define how to select the therapeutic support surface based on patient records. In the depicted embodiment, the first category of instructions is further sub-divided into sub-categories of instructions, each defining how to select the therapeutic support surface based on different aspects of patient records.

At block 212, therapeutic support surface distribution system 102 applies instructions that define how to select the therapeutic support surface based on the patient's medical records. In some embodiments, the instructions also define how to select the therapeutic support surface based on which medical condition(s) is patient is suffering from. For example, therapeutic support surface distribution system 102, in response to determining that the patient is suffering from a leg injury, selects a therapeutic support surface configured or configurable to provide support for the patient's injured leg. Alternatively, therapeutic support surface distribution system 102, in response to determining that the patient is suffering from a neck injury, selects another therapeutic support surface that is configured or configurable to support the patient's neck. In one or more of such embodiments, the instructions also define how to select the therapeutic support surface based on prior medical conditions of the patient. Continuing with the foregoing example, where therapeutic support surface distribution system 102, in response to determining that the patient is suffering from a neck injury, and in response to also determining that the patient previously suffered from a back injury, selects a therapeutic support surface that is configured or configurable to support the patient's back and neck. In some embodiments, the instructions also define how to select the therapeutic support surface based on the patient's demographics.

At block 214, therapeutic support surface distribution system 102 applies instructions that define how to select the therapeutic support surface based on the patient's preferences. In some embodiments, therapeutic support surface distribution system 102 complies with certain but not all preferences. In one or more of such embodiments, therapeutic support surface distribution system 102 assigns a weighted system to different patient preferences, where the weight of a preference is based on the amount of impact the preference has towards the patient's recovery. For example, a patient's preference on the type of mattress that alleviates the patient's recurring back pain is given a first weight that is greater than a patient's preference on the color of the therapeutic support surface. In one or more of such embodiments, therapeutic support surface distribution system 102 assigns a weighted system to different patient preferences, where the weight of the preference is based on whether the preference is incompatible with other rules for selecting the therapeutic support surface. For example, a patient's preference to select a model of a therapeutic support surface that fits into the designated location is given a first weight, whereas the patient's preference to select a second model that does not physically fit within the confines of the designated location is given a second weight that is less than the first weight or given no weight.

At block 216, therapeutic support surface distribution system 102 applies instructions that define how to select the therapeutic support surface based on the records of other patients. In some embodiments, therapeutic support surface distribution system 102 analyzes medical records and recovery times of other patients who suffered from similar or identical medical conditions. In one or more of such embodiments, therapeutic support surface distribution system 102 determines whether recovery times of other patients were less than the average recovery time, and selects a therapeutic support surface previously used by other patients that had improved the recovery times of the other patients. For example, where the patient is recovering from a hip replacement surgery, and therapeutic support surface distribution system 102 determines that ten patients who previously occupied a certain model of therapeutic support surface had an average recovery time that is less than the average recovery time by a threshold amount of time (e.g., a week, two weeks, or another period of time), then therapeutic support surface distribution system 102 would select the model of therapeutic support surface used by the other ten patients. In some embodiments, therapeutic support surface distribution system analyzes feedback of patients suffering from similar or identical medical conditions, and selects a therapeutic support surface having the highest average feedback or having an average feedback that is greater than a threshold feedback.

At block 230, therapeutic support surface distribution system 102 accesses a second category of instructions that define how to select the therapeutic support surface based on the location of the expected occupancy. At block 232, therapeutic support surface distribution system 102 applies instructions that define how to select the therapeutic support surface based on the type of the facility. For example, therapeutic support surface distribution system 102 is configured to select different therapeutic support surfaces based on whether the patient is expected to occupy the therapeutic support surface at an acute care facility (e.g., hospital), a post-acute care facility, or at home. In one more of such embodiments, therapeutic support surface distribution system 102 analyzes the floor plans of the facility and the available floor space of the designated location, and selects a therapeutic support surface that physically fits within the confines of the designated location. At block 234, therapeutic support surface distribution system 102 applies instructions that define how to select the therapeutic support surface based on the existing inventory at the facility. In some embodiments, where the facility has an existing inventory of therapeutic support surfaces, therapeutic support surface distribution system 102 determines whether one of the onsite therapeutic support surfaces satisfies most or all of the criteria. In one or more of such embodiments, where therapeutic support surface distribution system 102 determines that an onsite therapeutic support surface may be selected after certain modifications, therapeutic support surface distribution system 102 requests personnel (such as personnel of the facility or third-party personnel) to make the modifications to the onsite therapeutic support surface, and move the modified therapeutic support surface to the designated location. At block 236, therapeutic support surface distribution system 102 applies instructions that define how to select the therapeutic support surface based on the distance to the nearest therapeutic support surface storage location. In some embodiments, therapeutic support surface distribution system 102 selects a therapeutic support surface located at a storage location closest to the designated location, or is within a threshold distance (e.g., 25 miles, 50 miles, 100 miles, or another threshold distance) from the designated location.

At block 250, therapeutic support surface distribution system 102 accesses a third category of instructions that define how to select the therapeutic support surface based on the time and duration of the expected occupancy. At block 252, therapeutic support surface distribution system 102 applies instructions that define how to select the therapeutic support surface based on the estimated arrival time of the patient. In some embodiments, therapeutic support surface distribution system 102, in addition to determining the current availability of a therapeutic support surface, also selects a therapeutic support surface from an inventory of therapeutic support surfaces based on the amount of storage time to reduce or minimize the amount of time unused therapeutic support surfaces are stored in storage. In some embodiments, therapeutic support surface distribution system 102, in response to determining a delay in the estimated arrival time of the patient to the designated location, determines whether to hold the therapeutic support surface for the patient or to provide the therapeutic distribution surface to another patient that has a more urgent need than the patient, provided that therapeutic support surface distribution system 102 provides another therapeutic support surface to the patient prior to the new estimated arrival time. For example, where the patient was originally scheduled to arrive at the designated location on Feb. 1, 2021, but the arrival time was postponed to Feb. 10, 2021, and the therapeutic support surface may be provided to a second patient who is expected to occupy a therapeutic support surface starting on Feb. 5, 2021 at a second designated location, therapeutic support surface distribution system 102 determines whether the therapeutic support surface may be provided to the second patient. In one or more of such embodiments, therapeutic support surface distribution system 102 determines whether another therapeutic support surface may be provided to the patient on Feb. 10, 2021, and upon a determination that a second therapeutic support surface that is currently in use may be provided to the patient on Feb. 10, 2021, therapeutic support surface distribution system 102 dynamically reroutes delivery of the therapeutic support surface to the second designated location, and schedules cleaning and delivery of the second therapeutic support surface to the designated location by Feb. 10, 2021. At block 254, therapeutic support surface distribution system 102 applies instructions that define how to select the therapeutic support surface based on the patient's scheduled occupation period of time.

At block 256, therapeutic support surface distribution system 102 applies instructions that define how to select the therapeutic support surface based on delivery time. In some embodiments, therapeutic support surface distribution system 102 selects a therapeutic support surface that takes the least amount of time, or takes less than a threshold amount of time (e.g., one hour, one day, or another threshold of time) to deliver to the designated location. In some embodiments, where therapeutic support surface distribution system determines a potential delay in the delivery of the therapeutic support surface to the designated location, therapeutic support surface distribution system determines whether another therapeutic support surface may be delivered from another storage location within a threshold delivery time. For example, therapeutic support surface distribution system 102, in response to determining that an incoming snow storm would cause the delivery time to increase by more than a threshold period of time (e.g., one day), requests delivery of another therapeutic support surface from another storage location that takes less than the threshold delivery time. Similarly, where therapeutic support surface distribution system 102 determines an actual delay in the delivery of the therapeutic support surface to the designated location, therapeutic support surface distribution system 102 determines whether another therapeutic support surface may be delivered from another storage location within a threshold delivery time. For example, therapeutic support surface distribution system 102, in response to determining that a traffic accident is causing a delay by more than a threshold of time (e.g., five hours), requests delivery of another therapeutic support surface from another storage location that takes less than the threshold delivery time.

At block 258, therapeutic support surface distribution system 102 applies instructions that define how to select the therapeutic support surface to account for the installation time. In one or more of such embodiments, where therapeutic support surface distribution system 102 receives requests to modify certain components of a therapeutic support surface distribution, therapeutic support surface distribution system 102 requests personnel responsible for installation of the therapeutic support surface to complete installation and finish any modifications prior to the estimated arrival time of the patient.

At block 270, therapeutic support surface distribution system 102 accesses a fourth category of instructions that define how to select the therapeutic support surface based on cost. At block 272 therapeutic support surface distribution system 102 applies instructions that define how to select the therapeutic support surface based on the cost to the patient. In some embodiments, therapeutic support surface distribution system 102 analyzes the patient's insurance coverage and determines whether the patient's insurance covers some or all available therapeutic support surfaces, and the cost to the patient. In one or more of such embodiments, therapeutic support surface distribution system 102 selects the cheapest available therapeutic support surface or a therapeutic support surface that is within a threshold price range.

At block 274, therapeutic support surface distribution system 102 applies instructions that define how to select the therapeutic support surface based on the cost to the facility. In some embodiments, therapeutic support surface distribution system 102 analyzes the cost to the healthcare facility (such as, but not limited to, rental fee, insurance, delivery fee, and other fees charged to the healthcare facility), and determines which available therapeutic support surface to provide to the healthcare facility based on the cost to the facility. In one or more of such embodiments, therapeutic support surface distribution system 102 selects the least costly available therapeutic support surface or an available therapeutic support surface that in within a threshold cost range.

At block 276 therapeutic support surface distribution system 102 applies instructions that define how to select the therapeutic support surface based on the cost to the owner of the therapeutic support surface. In some embodiments, therapeutic support surface distribution system 102 analyzes the cost to the owner (such as, but not limited to, the cost to store, deliver, insurance, as well as other costs to the owner), and determines which available therapeutic support surface to provide to the healthcare facility based on the cost to the owner. In one or more of such embodiments, therapeutic support surface distribution system 102 is configured to reduce or minimize delivery time and storage time of therapeutic support surfaces, thereby reducing the cost to the owner.

At block 290, therapeutic support surface distribution system 102 accesses a fifth category of instructions that define how to select the therapeutic support surface based on user-selected criteria. At blocks 292 and 294, therapeutic support surface distribution system 102 applies instructions that define how to select the therapeutic support surface based on patient-designated criteria and care giver designated criteria, respectively. For example, where the patient designated criteria includes a request not to provide the patient with a certain model of therapeutic support surfaces, therapeutic support surface distribution system 102 will include the patient's request as a criterion for selecting a therapeutic support surface if there are available therapeutic support surfaces that also meet other criteria for selecting a therapeutic support surface for the patient.

In certain embodiments, where some instructions are incompatible with other instructions, therapeutic support surface distribution system 102 applies a weighted system on different instructions for selecting a therapeutic surface. For example, instructions on how to select the therapeutic support surface based on the patient's existing medical condition are given a first weight, instructions on how to select the therapeutic support surface based on cost to the facility are given a second weight that is less than the first weight, and instructions on how to select a therapeutic support surface based on aesthetics preferences of the patient are given a third weight that is less than the second weight, or given no weight.

In some embodiments, where therapeutic support surface distribution system 102 determines that multiple different models of therapeutic support surfaces are available for delivery, therapeutic support surface distribution system 102 provides a list of the available models of therapeutic support surfaces to the healthcare facility and/or to the patient. In one or more of such embodiments, therapeutic support surface distribution system 102 arranges available therapeutic support surfaces in a customizable order based on cost to the patient, cost to the healthcare facility, availability (e.g., number of identical therapeutic support surfaces in stock), patient rating, caregiver rating, as well as other features. In one or more of such embodiments, therapeutic support surface distribution system 102 ranks the available therapeutic support surfaces (e.g., based on best fit, customer feedback, or other types of metrics), and provides the healthcare facility and/or the patient with the ranking of the available therapeutic support surfaces. In one or more of such embodiments, where therapeutic support surface distribution system 102 subsequently receives a selection from the healthcare facility or the patient, therapeutic support surface distribution system 102 designates the selected therapeutic support surface for the patient as the available therapeutic support surface for delivery to the designated location.

Although the foregoing paragraphs describe five categories of instructions that define how to select the therapeutic support surface, the instructions may be organized into a different number of categories. Further, in some embodiments, where multiple instructions that define how to select the therapeutic support surface are applicable, therapeutic support surface distribution system 102 is operable to apply each applicable instruction and to determine how to select the therapeutic support surface based on all applicable instructions. Further, therapeutic support surface distribution system 102 is operable to execute the instructions described in the foregoing paragraph to simultaneously select multiple therapeutic support surfaces for different patients.

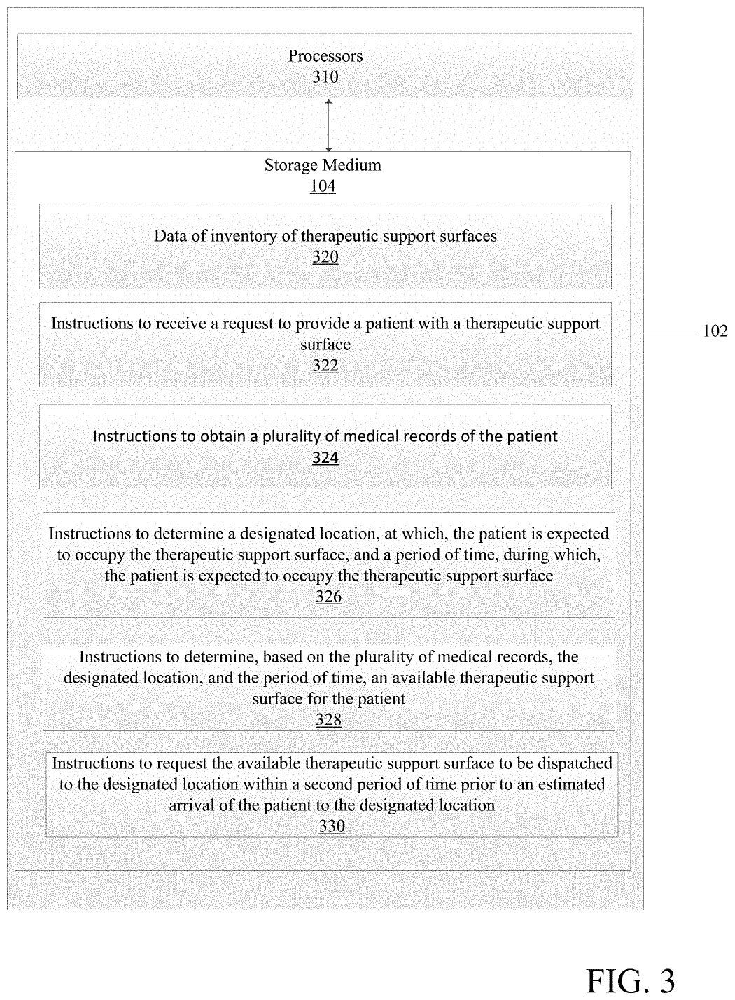

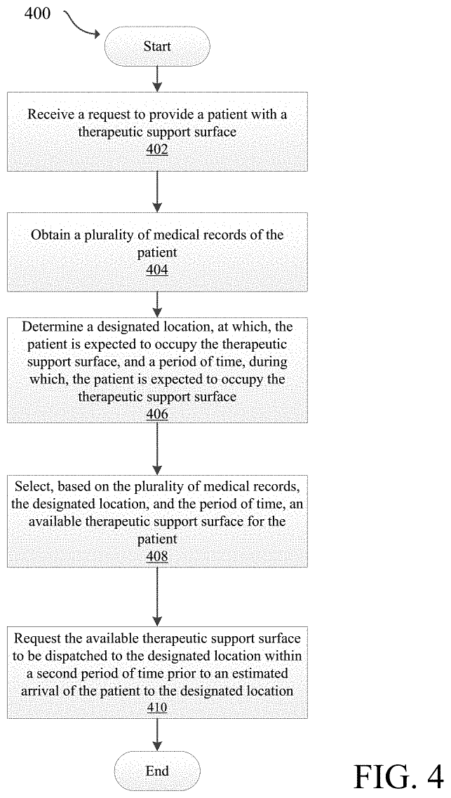

FIG. 3 is a system diagram of therapeutic support surface distribution system 102 of FIGS. 1A and 1B in accordance with one embodiment. Therapeutic support surface distribution system 102 includes or is communicatively connected to storage medium 104 and processors 310. Data indicative of the therapeutic support surfaces, including the specification and modification of different therapeutic support surfaces, the location of the therapeutic support surfaces, installation guides, as well as other information regarding the therapeutic support surfaces are stored at location 320 of storage medium 104. In some embodiments, certain patient data are also stored in storage medium 104 provided storage of such data is consented by applicable patients and complies with the law. Instructions to receive a request to provide a patient with a therapeutic support surface are stored at location 322. Further, instructions to obtain a plurality of medical records of the patient are stored at location 324. Further, instructions to determine a designated location, at which the patient is expected to occupy the therapeutic support surface, and a period of time during which the patient is expected to occupy the therapeutic support surface are stored at location 326. Further, instructions to determine, based on the plurality of medical records, the designated location, and the period of time, an available therapeutic support surface for the patient are stored at location 328. Further, instructions to request the available therapeutic support surface to be dispatched to the designated location within a second period of time prior to an estimated arrival of the patient to the designated location are stored at location 330. Further, instructions to perform operations described herein and shown in at least FIGS. 2 and 4 are also stored in storage medium 104.

FIG. 4 is a flow chart illustrating a process 400 to dynamically distribute therapeutic support surfaces in accordance with one embodiment. Although the operations in process 400 are shown in a particular sequence, certain operations may be performed in different sequences or at the same time where feasible. Further, although process 400 is described to be performed by processors of therapeutic support surface distribution system 102 of FIGS. 1A and 1B, it is understood that processors of other therapeutic support surface distribution systems are also operable to perform process 400.

At block 402, a therapeutic support surface distribution system, such as therapeutic support surface distribution system 102 of FIGS. 1A and 1B receives a request to provide a patient with a therapeutic support surface. FIGS. 1A and 1B, for example, illustrate therapeutic support surface distribution system 102 receiving a request from healthcare facility system 110 to provide a patient with a therapeutic support surface. At block 404, the therapeutic support surface distribution system 102 obtains a plurality of medical records of the patient. In the embodiment of FIGS. 1A and 1B, healthcare facility system 110 also provides therapeutic support surface distribution system 102 with the medical records of the patient. In some embodiments, some of the patient's medical records are also provided by third-party system 130 of FIGS. 1A and 1B, or by user 121 of FIG. 1B via electronic device 120 of FIG. 1B.

At block 406, the therapeutic support surface distribution system 102 determines a designated location at which the patient is expected to occupy the therapeutic support surface, and a period of time during which the patient is expected to occupy the therapeutic support surface. In the embodiment of FIGS. 1A and 1B, the designated location, and the period of time the patient is expected to occupy the therapeutic support surface are provided by healthcare facility system 110 to therapeutic support surface distribution system 102.

At block 408, the therapeutic support surface distribution system, selects, based on the plurality of medical records, the designated location, and the period of time, an available therapeutic support surface for the patient. FIG. 2, for example, illustrates a tree diagram illustrating a process 200 to select a therapeutic support surface in accordance with and based on categories and instructions described herein. In some embodiments, where the therapeutic support surface distribution system determines one or more medical conditions affecting the patient, the therapeutic support surface distribution system also selects the available therapeutic support surface that alleviates the one or more medical conditions of the patient. In one or more of such embodiments, the therapeutic support surface distribution system identifies one or more therapeutic support surfaces occupied by one or more patients having similar medical conditions, and selects the available therapeutic support surface from among the one or more therapeutic support surfaces occupied by the one or more patients. In some embodiments, the therapeutic support surface distribution system also requests one or more modifications of one or more components (e.g., adjust the cushion thickness, adjust the headrest, and other types of available modifications) of the available therapeutic support surface to alleviate one or more conditions of the patient. In some embodiments, the therapeutic support surface distribution system receives an user-inputted criterion (e.g., from the patient or a caregiver of the patient) for selecting the available therapeutic support surface, and selects the available therapeutic support surface based on the user-provided criterion.

At block 410, the therapeutic support surface distribution system requests the available therapeutic support surface to be dispatched to the designated location within a second period of time prior to an estimated arrival of the patient to the designated location. In some embodiments, the therapeutic support surface distribution system determines a time from which the patient began occupying the available therapeutic support surface, and schedules for retrieval of the retrieval of the available therapeutic support surface from the designated location after the scheduled occupancy time has passed. For example, where the therapeutic support surface distribution system determines that the patient began occupying the available therapeutic support surface from 12 PM Central Time on Feb. 14, 2021 and is scheduled to occupy the available therapeutic support surface for two week, the therapeutic support surface distribution system schedules the available therapeutic support surface to be retrieved at or shortly after 12 PM Central Time on Feb. 28, 2021. In some embodiments, the therapeutic support surface distribution system determines a new period of time the patient is expected to occupy the available therapeutic support surface, and reschedules retrieval of the available therapeutic support surface from the designated location after the new period of time from the time the patient began occupying the available therapeutic support surface has passed.

In some embodiments, while the patient is occupying the available therapeutic support surface, the therapeutic support surface distribution system receives a second request to provide a second patient with a second therapeutic support surface. In one or more of such embodiments, the therapeutic support surface distribution system, in response to receiving such request, obtains the medical records of the second patient, and determining a second designated location at which the second patient is expected to occupy the second therapeutic support surface, and a period of time during which the second patient is expected to occupy the second therapeutic support surface. Further, the therapeutic support surface distribution system, in response to a determination to provide the available therapeutic support surface to the second patient, requests the available therapeutic support surface to be transported to the second designated location within a period of time prior to an estimated arrival of the second patient to the second designated location. In some embodiments, the therapeutic support surface distribution system determines a status of a condition of the patient while occupying the available therapeutic support surface, and in response to a determination of an improvement of the condition while occupying the available therapeutic support surface, designates a model of the available therapeutic support surface for occupancy by future patients that experience a similar condition as the condition of the patient.

Process 400 allows therapeutic support surfaces to be dynamically requested and provided to a designated location at a healthcare facility prior to an expected arrival time of a patient to the designated location. Process 400 reduces a need for an user, such as a patient or a caregiver, to communicate with a therapeutic support surface distribution system to request a therapeutic support surface, thereby reducing the strain on existing network, which frees up communications systems, reduces bandwidth on networks, and preserves energy. Moreover, by automating the process to request therapeutic support surfaces, process 400 not only reduces the cognitive burden on the user, but also reduces or eliminates human-related error, thereby reducing wait time for life saving medical equipment, and improving the patient recovery periods. Further, after a request for a therapeutic support surface is made, process 400 also reduces the cognitive burden on an user by automatically providing the status updates of inventory, transit, delivery, and installation of the requested therapeutic support surface, thereby creating a more efficient human-machine interface. Moreover, by dynamically providing healthcare systems with status updates on requested therapeutic support surfaces, process 400 also reduces the strain on electronic systems of healthcare facilities tasked to procure therapeutic support surfaces, thereby reducing the strain on existing network, which frees up communications systems, reduces bandwidth on networks, and preserves energy.

The above-disclosed embodiments have been presented for purposes of illustration and to enable one of ordinary skill in the art to practice the disclosure, but the disclosure is not intended to be exhaustive or limited to the forms disclosed. Many insubstantial modifications and variations will be apparent to those of ordinary skill in the art without departing from the scope and spirit of the disclosure. The scope of the claims is intended to broadly cover the disclosed embodiments and any such modification.

As used herein, the singular forms “a,” “an,” and “the” are intended to include the plural forms as well, unless the context clearly indicates otherwise. It will be further understood that the terms “comprise” and/or “comprising,” when used in this specification and/or the claims, specify the presence of stated features, steps, operations, elements, and/or components, but do not preclude the presence or addition of one or more other features, steps, operations, elements, components, and/or groups thereof. In addition, the steps and components described in the above embodiments and figures are merely illustrative and do not imply that any particular step or component is a requirement of a claimed embodiment.

What is claimed is:

1. A method to dynamically distribute therapeutic support surfaces, comprising:
receiving a request to provide a patient with a therapeutic support surface;
obtaining a plurality of medical records of the patient;
determining a designated location, at which, the patient is expected to occupy the therapeutic support surface, and a period of time, during which, the patient is expected to occupy the therapeutic support surface;
selecting, based on the plurality of medical records, the designated location, and the period of time, an available therapeutic support surface for the patient; and
requesting the available therapeutic support surface to be transported to the designated location within a second period of time prior to an estimated arrival of the patient to the designated location.

2. The method of claim 1, further comprising determining one or more medical conditions affecting the patient based on the plurality of medical records, wherein selecting the available therapeutic support surface comprises selecting, based on the one or more medical conditions, a therapeutic support surface that alleviates one or more of the one or more medical conditions.

3. The method of claim 2, further comprising:
identifying one or more therapeutic support surfaces occupied by one or more patients having a similar medical condition as a medical condition of the one or more medical conditions affecting the patient; and
selecting, from among the one or more therapeutic support surfaces, the available therapeutic support surface for the patient.

4. The method of claim 3, further comprising:
determining one therapeutic support surface of the one or more therapeutic support surfaces that improved recovery time of the one or more patients by a threshold amount of time; and
designating the one therapeutic support surface as the available therapeutic support surface.

5. The method of claim 2, further comprising modifying one or more components of the available therapeutic support surface to alleviate the one or more medical conditions.

6. The method of claim 1, further comprising:
obtaining insurance information of the patient; and
selecting, based on the insurance information, the available therapeutic support surface for the patient.

7. The method of claim 1, further comprising:
determining, from an inventory of available therapeutic support surfaces, one or more therapeutic support surfaces that cost less than a threshold amount to be transported to the designated location; and
designating one of the one or more therapeutic support surfaces as the available therapeutic support surface.

8. The method of claim 1, further comprising:
determining, from an inventory of available therapeutic support surfaces, one or more therapeutic support surfaces that are stored at a location within a threshold range of the designated location; and
designating one of the one or more therapeutic support surfaces as the available therapeutic support surface.

9. The method of claim 1, further comprising:
receiving an user-inputted criterion for determining the available therapeutic support surface; and
selecting, based on the user-inputted criterion, the available therapeutic support surface for the patient.

10. The method of claim 9, further comprising:
determining, based on the plurality of medical records, the designated location, and the period of time, a set of user-selectable criteria for selecting the available therapeutic support surface; and
providing the set of user-selectable criteria to an electronic device of an user,
wherein the user-inputted criterion is selected from the set of user-selectable criteria.

11. The method of claim 1, further comprising requesting installation of the available therapeutic support surface to be complete prior to the estimated arrival of the patient.

12. The method of claim 1, further comprising:
determining a time from which the patient began occupying the available therapeutic support surface; and
scheduling retrieval of the available therapeutic support surface from the designated location after the period of time from the time the patient began occupying the available therapeutic support surface has passed.

13. The method of claim 12, further comprising:
determining a new period of time the patient is expected to occupy the available therapeutic support surface; and
rescheduling retrieval of the available therapeutic support surface from the designated location after the new period of time from the time the patient began occupying the available therapeutic support surface has passed.

14. The method of claim 1, further comprising:
while the patient is occupying the available therapeutic support surface, receiving a second request to provide a second patient with a second therapeutic support surface;
obtaining a plurality of medical records of the second patient;
determining a second designated location, at which, the second patient is expected to occupy the second therapeutic support surface, and a third period of time, during which, the second patient is expected to occupy the second therapeutic support surface; and
in response to a determination to provide the available therapeutic support surface to the second patient, requesting the available therapeutic support surface to be transported to the second designated location within a fourth period of time prior to an estimated arrival of the second patient to the second designated location.

15. The method of claim 1, further comprising:
determining a status of a condition of the patient while occupying the available therapeutic support surface; and
in response to a determination of an improvement of the condition while occupying the available therapeutic support surface, designating a model of the available therapeutic support surface for occupancy by future patients that experience a similar condition as the condition of the patient.

16. A system to dynamically distribute therapeutic support surfaces, comprising:
a storage medium; and
one or more processors configured to:
receive a request to provide a patient with a therapeutic support surface;
obtain a plurality of medical records of the patient;
determine a designated location, at which, the patient is expected to occupy the therapeutic support surface, and a period of time, during which, the patient is expected to occupy the therapeutic support surface;
select, based on the plurality of medical records, the designated location, and the period of time, an available therapeutic support surface for the patient;
request the available therapeutic support surface to be transported to the designated location within a second period of time prior to an estimated arrival of the patient to the designated location; and
request installation of the available therapeutic support surface to be complete prior to the estimated arrival of the patient.

17. The system of claim 16, wherein the one or more processors are configured to determine one or more medical conditions affecting the patient based on the plurality of medical records, and select the available therapeutic support surface based on the one or more medical conditions, a therapeutic support surface that alleviates one or more of the one or more medical conditions.

18. The system of claim 16, wherein the one or more processors are further configured to:
determine a time from which the patient began occupying the available therapeutic support surface; and
schedule retrieval of the available therapeutic support surface from the designated location after the period of time from the time the patient began occupying the available therapeutic support surface has passed.

19. A non-transitory machine-readable medium comprising instructions stored therein, which when executed by one or more processors, cause the one or more processors to perform operations comprising:
receiving a request to provide a patient with a therapeutic support surface;
obtaining a plurality of medical records of the patient;
determining a designated location, at which, the patient is expected to occupy the therapeutic support surface, and a period of time, during which, the patient is expected to occupy the therapeutic support surface;
selecting, based on the plurality of medical records, the designated location, and the period of time, an available therapeutic support surface for the patient;
requesting the available therapeutic support surface to be transported to the designated location within a second period of time prior to an estimated arrival of the patient to the designated location;
requesting installation of the available therapeutic support surface to be complete prior to the estimated arrival of the patient;
determining a time from which the patient began occupying the available therapeutic support surface; and
scheduling retrieval of the available therapeutic support surface from the designated location after the period of time from the time the patient began occupying the available therapeutic support surface has passed.

20. The non-transitory machine-readable medium of claim 19, further comprising instructions stored therein, which when executed by one or more processors, cause the one or more processors to perform operations comprising:
while the patient is occupying the available therapeutic support surface, receiving a second request to provide a second patient with a second therapeutic support surface;
obtaining a plurality of medical records of the second patient;
determining a second designated location, at which, the second patient is expected to occupy the second therapeutic support surface, and a third period of time, during which, the second patient is expected to occupy the second therapeutic support surface; and
in response to a determination to provide the available therapeutic support surface to the second patient, requesting the available therapeutic support surface to be transported to the second designated location within a fourth period of time prior to an estimated arrival of the second patient to the second designated location.
